(19) United States
(12) Patent Application Publication
(10) Pub. No.: US 2026/0224305 A1
(43) Pub. Date: Aug. 6, 2026

(54) Surgical System for Minimally Invasive Robotic Surgery

(71) Applicant: Deutsches Zentrum fur Luft- und Raumfahrt e.V., Bonn (DE)

(72) Inventor: Christopher Schlenk, München (DE)

(21) Appl. No.: 19/147,407

(22) PCT Filed: Feb. 28, 2024
(86) PCT No.: PCT/EP2024/055136

§ 371 Date: Jul. 11, 2025

(30) Foreign Application Priority Data
Feb. 28, 2023 (DE) 10 2023 104 936.4

(51) Int. Cl. A61B 34/30; A61B 1/00; A61B 1/015

(52) U.S. Cl. CPC A61B 34/30; A61B 1/00039; A61B 1/00133; A61B 1/00149; A61B 1/015; A61B 2034/301

(57) ABSTRACT

The invention relates to a surgical system for minimally invasive robotic surgery, having: a holder arm having a mount for the handle unit of a conventional endoscope, and having a patient-side unit which is fastenable in the vicinity of the patient and enables defined positioning of the patient access, characterized by a fluid container, and a pump unit for pumping an irrigation fluid into the patient's body.

### CROSS-REFERENCE TO RELATED APPLICATIONS

This application is the United States national phase of International Patent Application No. PCT/EP2024/055136 filed Feb. 28, 2024, and claims priority to German Patent Application No. 10 2023 104 936.4 filed Feb. 28, 2023, the disclosures of which are hereby incorporated by reference in their entireties.

### BACKGROUND OF THE INVENTION

### Field of the Invention

The invention relates to a surgical system for minimally invasive robotic surgery.

### Description of Related Art

In endoscopy, in particular ureteroscopy (endoscopic procedures in the urethra, bladder, ureter and kidney), endoscopes are used to perform diagnostic tasks (e.g. optical examination of organs) or manipulations (e.g. taking biopsies, removing foreign bodies such as kidney stones) within hollow organs. These can either be rigid (“rigid endoscopes”) or can be angled in at least one degree of freedom (“flexible endoscopes”). During surgery, the endoscopes are typically guided manually by the surgeon, which is technically challenging, especially with flexible endoscopes: The surgeon holds the handle of the flexible endoscope in one hand and uses a lever on the handle to activate the angulation of the endoscope tip and the rotation of the endoscope around its longitudinal axis by turning the handle, while with the other hand, he controls the advance of the flexible endoscope shaft into the patient.

Commercially available accessories for flexible endoscopes such as the LithoVue Empower (Boston Scientific, Marlborough, MA, USA) allow the surgeon to perform individual steps (such as stone retrieval) alone. Such accessory components are compact and do not impair the haptic feedback of the surgeon, as he still activates all three endoscopic degrees of freedom (advancement, rotation around the longitudinal axis and angulation) by hand.

The Avicenna Roboflex is currently a commercially available surgical system for flexible ureteroscopy: The flexible endoscope is docked onto a holding arm located on a cart, which also offers actuation options for angling the endoscope tip and moving the laser fiber.

Intuitive Ion is a surgical system for minimally invasive peripheral lung biopsies that can navigate a flexible bronchoscope telemanipulated through the bronchi of the lung.

Auris Monarch is another surgical system for peripheral bronchoscopy.

Hansen Medical has developed two systems for catheter manipulation, the Magellan Surgical System and the Sensei Surgical System. The Magellan was designed for peripheral, vascular robotic interventions, the Sensei for interventional electrophysiological interventions.

Corindus CorPath GRX is a robotic system for the telemanipulated positioning of catheters in vascular surgery.

Another telemanipulated system for urology, the Zamenix R (ROEN Surgical Inc., Daejeon, Korea), is currently being launched as a commercial product. Apart from the design of the surgeon controller (see patent WO002020218678A1), its basic concept is very similar to the Avicenna Roboflex.

The robotic systems for controlling flexible endoscopes in research papers can be classified into two categories:

Dockable actuation units [sources 1-6 see below] actuate some or all degrees of freedom on the endoscope handle and are either held in the surgeon's hand together with the endoscope or attached to a passive holding arm. These units are compact, but most do not activate all endoscope degrees of freedom and haptic feedback is impaired in the activated endoscope degrees of freedom. Hand-held systems increase the weight to be carried by the surgeon. Attachment to a passive support arm makes system handling cumbersome in interventions that require frequent repositioning of the endoscope handle: Each time the endoscope is repositioned, the combined weight of the endoscope, holding arm and actuation unit must be moved, wherein the passive holding arm restricts the movement options.

### Sources

[1] Olds, K., Hillel, A., Kriss, J., Nair, A., Kim, H., Cha, E., Curry, M., Akst, L., Yung, R., Richmon, J., Taylor, R.: A robotic assistant for trans-oral surgery: the robotic endolaryngeal flexible (robo-ELF) scope. Journal of Robotic Surgery 6 (1), 13-18 (2011). https://doi.org/10.1007/s11701-011-0329-9
[2] Fang, C., Cesmeci, D., Gumprecht, J. D. J., Krause, E.-M., Strauss, G., Lueth, T. C.: Amotorized hand-held flexible rhino endoscope in ENT diagnoses and its clinical experiences. In: IEEE (ed.) 2012 4th IEEE RAS & EMBS International Conference on Biomedical Robotics and Biomechatronics (BioRob), pp. 853-858 (2012). https://doi.org/10.1109/biorob.2012.6290784
[3] Zhang, L. A., Khare, R., Wilson, E., Wang, S. X., Peters, C. A., Cleary, K.: Robotic assistance for manipulating a flexible endoscope. In: IEEE (ed.) 2014 IEEE International Conference on Robotics and Automation (ICRA), pp. 5380-5385 (2014). https://doi.org/10.1109/icra.2014.6907650
[4] Ruiter, J. G., Bonnema, G. M., van der Voort, M. C., Broeders, I. A. M. J.: Robotic control of a traditional flexible endoscope for therapy. Journal of Robotic Surgery 7 (3), 227-234 (2013). https://doi.org/10.1007/s11701-013-0405-4
[5] Iwasa, T., Nakadate, R., Onogi, S., Okamoto, Y., Arata, J., Oguri, S., Ogino, H., Ihara, E., Ohuchida, K., Akahoshi, T., Ikeda, T., Ogawa, Y., Hashizume, M.: A new robotic-assisted flexible endoscope with singlehand control: endoscopic submucosal dissection in the ex vivo porcine stomach. Surgical Endoscopy 32 (7), 3386-3392 (2018). https://doi.org/10.1007/s00464-018-6188-y
[6] Lee, D.-H., Cheon, B., Kim, J., Kwon, D.-S.: easyEndo robotic endoscopy system: Development and usability test in a randomized controlled trial with novices and physicians. The International Journal of Medical Robotics and Computer Assisted Surgery 17 (1), 1-14 (2020). https://doi.org/10.1002/rcs.2158

Teleoperation systems [7-12] enable remote control of the endoscope and its end effectors in all degrees of freedom from a surgeon's console. The surgeon no longer has to hold the weight of the components. However, the haptic feedback is impaired in the activated degrees of freedom or technically complex and expensive force or torque sensors must be provided. In addition, the combined footprint of the robot and surgeon's console is large and a second surgeon is required at the operating table, for example to insert the endoscope into the patient. An intraoperative change to manual endoscopy (e.g. because haptic feedback is advantageous or a malfunction occurs) is time-consuming, as the surgeon has to dress in sterile gowns, the endoscope has to be removed from the robotic system and the robot has to be removed from the patient.

### Sources

[7] Desai, M. M., Grover, R., Aron, M., Ganpule, A., Joshi, S. S., Desai, M. R., Gill, I. S.: Robotic flexible ureteroscopy for renal calculi: Initial clinical experience. Journal of Urology 186 (2), 563-568 (2011). https://doi.org/10.1016/1.juro.2011.03.128
[8] Rassweiler, J., Fiedler, M., Charalampogiannis, N., Kabakci, A. S., Saglam, R., Klein, J.-T.: Robot-assisted flexible ureteroscopy: an update. Urolithiasis 46 (1), 69-77 (2017). https://doi.org/10.1007/s00240-017-1024-8
[9] Geavlete, P., Saglam, R., Georgescu, D., Mult escu, R., Iordache, V., Kabakci, A. S., Ene, C., Geavlete, B.: Robotic flexible ureteroscopy versus classic flexible ureteroscopy in renal stones: the initial Romanian experience. Chirurgia 111, 326-329 (2016)
[10] Shu, X., Chen, Q., Xie, L.: A novel robotic system for flexible ureteroscopy. The International Journal of Medical Robotics and Computer Assisted Surgery 17 (1), 1-11 (2020). https://doi.org/10.1002/rcs.2191
[11] Zhao, J., Li, J., Cui, L., Shi, C., Wei, G.: Design and performance investigation of a robot-assisted flexible ureteroscopy system. Applied Bionics and Biomechanics 2021, 1-13 (2021). https://doi.org/10.1155/2021/6911202
[12] Park, J., Gwak, C. H., Kim, D., Shin, J. H., Lim, B., Kim, J., Cheon, B., Han, J., Kwon, D.-S., Park, H. K.: The usefulness and ergonomics of a new robotic system for flexible ureteroscopy and laser lithotripsy for treating renal stones. Investigative and Clinical Urology 63 (6), 647 (2022). https://doi.org/10.4111/icu.20220237

US 2012/0065470 A1 describes a robotic system for guiding a commercial flexible endoscope, in particular in laryngology. Here, the flexible endoscope is placed in a suitable holder and the actuating element for bending the endoscope tip is placed in a clamp that can activate the actuating element. The entire holder can be rotated around the longitudinal axis of the endoscope and moved along the longitudinal axis of the endoscope using appropriate drives. The drives for the three degrees of freedom are located either in the immediate vicinity of the respective mechanisms or together in a motor housing, with the rotation and angular movement being transmitted via Bowden cables. The robotic system is controlled via a compact control unit with two joysticks (one with one degree of freedom, one with two degrees of freedom), which can either be positioned on a suitable surface or attached to the side rails of the operating table. The robotic system is connected to the side rail of the operating table via a passive stand and roughly positioned.

WO 2013/029 045 A1 describes an endoscope adapter consisting of a holder for the flexible endoscope and a manipulation mechanism which optionally moves the flexible endoscope shaft and/or a tool to be inserted into the working channel of the endoscope in the axial direction. For this purpose, the manipulation mechanism uses rollers, wherein at least one roller is pressed against the endoscope shaft/tool by a spring. It is optionally possible to drive at least one of the two rollers in order to actively control the movement. In one embodiment (FIG. 12), it is proposed to attach the manipulation mechanism to the patient.

WO 2019 139 941 A1 describes an adapter with which a rigid endoscope can be attached to the instrument interface of a medical robot in minimally invasive surgery. This adapter makes it possible to convert the rotation of an output of the instrument drive unit (for faster rotation) or alternatively the rotation of the entire instrument drive unit by a drive in the instrument holder (for slower rotation) into a rotation of the endoscope around its longitudinal axis. It is possible to use various commercially available standalone endoscopes with the adapter (the mounting shells for the endoscope may have to be replaced). Depending on the version, the endoscope is permanently installed in the adapter (FIGS. 3-6) or can be removed from it after opening a latch (FIGS. 8-11; FIGS. 21A-24).

U.S. Pat. No. 10,219,867 B2 describes the Avicenna Roboflex Surgical System: Various commercially available flexible endoscopes can be attached to a holder on the end effector of a robot positioned on a trolley. This holder can be moved back and forth in the direction of the endoscope axis and rotated around the endoscope axis. Furthermore, the holder contains a mechanism for actuating the degree of angular freedom of the flexible endoscope. In addition, mechanisms for activating auxiliary tools (laser fiber, pliers, recovery baskets, etc.) and a pump unit for controlling the flushing of the working channel are available. The system is telemanipulated by the surgeon from a console using two force feedback joysticks (one allows movements back and forth and rotations around the joystick axis, the second has a lever for coarse control of the endoscope angulation), an adjusting wheel for fine control of the endoscope angulation, foot pedals (e.g. for laser fiber and fluoroscopy) and a touch screen. Some safety functions are integrated (laser cannot be fired in the working channel of the endoscope, endoscope is straightened when the laser fiber is inserted), as well as autonomy functions (compensation of the patient's breathing movement by translational movement of the endoscope).

U.S. Pat. No. 9,763,741 B2 describes a robotic system for telemanipulation of a flexible endoscope. The endoscope is an instrument specially designed for this robotic system, which is attached to a drive unit that can be positioned by a holding arm. While the robot performs the translation and rotation of the endoscope around its longitudinal axis, the drive unit actuates the bending of the endoscope tip. The flexible endoscope shaft is guided by a rigid sheath guided by a second holding arm, into which sheath the endoscope shaft is inserted.

US 2017/0119412 A1 describes the guidance of a recovery basket by means of holding arms that can be moved under remote control from a surgeon's console or in hands-on mode. If the recovery basket is pulled together to catch an object, the robots automatically adjust the position of the recovery basket so that the object remains in the center of the recovery basket. As soon as the object has been caught, it can be shredded (by laser, fluid or mechanically) and aspirated through a central working channel while still in the basket.

US 2018/0092517 A1 describes a calibration method for flexible endoscopes in which the robotic system moves the endoscope to various target positions and receives feedback on the actual endoscope position via suitable sensors (e.g. electromagnetic sensor systems, cameras, fiber optic sensors). Based on this, correction factors for endoscope actuation are determined and saved. These can depend on various factors (e.g. activated cables, length of the endoscope tip outside the sheath, rotation of the endoscope in relation to the sheath) and can be stored in a calibration matrix. By integrating strain gauges into the instrument drive unit, the cable forces acting on the endoscope can also be monitored.

US 2019/0191967 A1 describes a robotic telemanipulation system with optional haptic feedback, in which the robotic instruments (consisting of holding arm and end effector) and the flexible endoscope for imaging are guided through the working channels of a flexible transport endoscope. The transport endoscope can be attached to a docking station during the surgical procedure. The degrees of freedom of the robotic instruments are activated by a common motor box while an endoscope support system remotely controls insufflation, suction and irrigation of the transport endoscope. The endoscope for imaging and the robotic instruments can be moved as a whole along their longitudinal axis and rotated around their longitudinal axis. To enable precise motion transmission from the motor unit to the robotic instruments, the pretension of the transmitting wire cables can be set automatically at system startup or intraoperatively.

German Patent Application 10 2019 134 352.6 describes a surgical robot for endoscopic applications in very general terms. Here, a flexible endoscope with its handle unit is detachably attached to an instrument base plate, while the movement and the advancement of the shaft is effected by a robotic arm. The degree of angular freedom of the endoscope is controlled by another actuator. Various possible assistance functions are described (gravity compensation, automatic advancement and retraction of the endoscope, automatic change of instruments such as laser fiber and recovery basket, motion compensation for patient movement, automatic orientation of the image, mapping, display of additional functions via augmented reality). The entire system is mounted on a movable platform so as to be mobile.

German Patent Application 10 2022 118 388.2 describes a system for the manipulation of flexible endoscopes with a holding arm, an attachment for the endoscope handle unit on the holding arm, a patient-side unit and an output unit for the system status.

Manual handling of the endoscope has various disadvantages:
Physical exertion due to the weight of the handle
Unergonomic hand position due to twisting of the handle and/or actuation of the control elements. In some cases, the movement possibilities of the endoscope inside the body are limited by the movement possibilities of the human hand.
Surgeon stands in the area of the X-ray machine during intraoperative X-rays. On the one hand, this makes it necessary to wear a lead vest and, on the other hand, increases the X-ray exposure of the surgeon.
Second surgeon required.
   Confined working conditions, as the available space (typically between the patient's spread legs during ureteroscopic procedures) is limited.
   Complex coordination: When using a tool such as a laser fiber or a retrieval basket, the movements of the endoscope and tool must be coordinated, which requires good coordination between the two surgeons.

The commercially available robotic solutions also have disadvantages:
Pure telemanipulation systems:
   Conversion to manual surgery only possible with difficulty
   Loss of surgical dexterity when moving endoscopes/catheters
   Repeated insertion and removal of the endoscope during stone retrieval in urology requires support from sterile personnel at the operating table
Highly specialized systems:
   Surgical systems customized for a specific application=>particularly interesting for large clinics with high treatment numbers
   Apart from Roboflex Avicenna: Use of special robotic instruments, which must be purchased in addition to the manual instruments

The publications mentioned have the following disadvantages with regard to the guidance of flexible endoscopes:

US 2012/0065470A1 : The activated degrees of freedom only allow small movements (especially during translation); for larger movements, the ports on the passive stand must be opened, the system components repositioned and the ports closed again. Intraoperative movement of the endoscope by hand is not easily possible, as removing the endoscope from the holder is complex (opening the holder and opening the clamp for the actuation lever) and there are no corresponding sensors to enable hands-on control of the system.

WO 2013/029 045A1 : The roller mechanism shown can actuate the translation of the flexible endoscope shaft or the tool in the working channel of the flexible endoscope shaft. However, the rotation of the endoscope around its longitudinal axis cannot be actuated with the mechanism described. However, this is indispensable, especially for smaller endoscopes that can only be angled in one plane (e.g. ureteroscopes), in order to be able to perform all the desired manipulation tasks inside the patient. Furthermore, it is apparently not possible to quickly release the frictional connection between the endoscope shaft and the rollers, as the rollers are spring-loaded. This means that the surgeon cannot advance the endoscope manually using his dexterity.

The adapter described in WO 2019 139 941 A1 is designed for rigid endoscopes. For this reason, it does not allow an actuation of the endoscope angling. On the other hand, a robot with such an adapter can position and align the handle of a flexible endoscope in space, but not the endoscope tip, as there is no guide for the flexible endoscope shaft and therefore no unique transmission of the movement of the handle to the movement of the endoscope tip.

The Avicenna Roboflex system described in U.S. Pat. No. 10,219,867 B2 has the above-mentioned disadvantages of robotic systems.

The robotic system for interventions with flexible endoscopes described in U.S. Pat. No. 9,763,741 B2, US 2017/0119412 A1 and US 2018/0092517 A1 also has the above-mentioned disadvantages of robotic systems. Furthermore, the use of specialized instruments and the high technical complexity of the system shown (three holding arms required to perform an endoscopic procedure in the kidney) are likely to make it considerably more difficult to use the system economically, particularly in smaller clinics with low case numbers.

Patent US 2019/0191967 A1 focuses on the actuation of the imaging endoscope and the robotic instruments, both of which are only designed for use with this system. The transport endoscope is still controlled manually, and the flexible shaft is only fixed at the position of the handle and patient access.

Compared to German Patent Application 10 2019 134 352.6, the present solution further develops the mechanical design of the system. In particular, the design of the attachment of the endoscope handle to the holding arm, the design of the port attachment at the access to the patient and the possible integration of virtual fixtures to support the surgeon should be mentioned here.

Compared to patent application 10 2021 114 429.9, the present solution represents a technically simplified approach in which the possibility of telemanipulation is eliminated due to the lack of actuation of the endoscope degrees of freedom. The positioning of the endoscopic end effectors and a control unit for controlling external devices close to the handle allows the surgeon to ergonomically control the system functions required during surgery. Virtual fixtures can support the surgeon intraoperatively, but can also be used in the training of young surgeons.

### SUMMARY OF THE INVENTION

It is an object of the invention to provide a surgical system for minimally invasive robotic surgery that enables simplified handling of an endoscope.

According to the invention, the object is achieved by the features as described herein.

The surgical system according to the invention for minimally invasive robotic surgery comprises a holding arm which has a holder for the handle unit of a conventional endoscope. Furthermore, a patient-side unit is provided that can be attached close to the patient and enables defined positioning of the patient access. Moreover, a fluid container and a pump unit are provided for pumping an irrigation fluid into the patient's body. Preferably, the pump unit can be operated by the surgeon operating the endoscope with the same hand.

For this purpose, it is preferred that the endoscope has at least one actuation button for actuating the pump unit and/or a laser. For example, if the endoscope has an actuation button for actuating the pump unit, the laser can be actuated via another input device, such as a foot pedal. Alternatively, the laser can be operated via the operating button on the endoscope and the pump unit via the other operating device.

For example, a tube from the outlet side of the pump unit can be connected to the working channel of a ureteroscope, e.g. via a three-way valve. The hoses are typically equipped with Luer lock connections for this purpose.

The present invention enables a surgeon to grasp the endoscope at its control unit with one hand and to carry out all necessary input commands there. With the second hand, the surgeon can grasp and guide the flexible shaft of the endoscope as usual.

A particular advantage of the present invention is the possibility of operating the necessary additional devices during the surgical procedure (e.g. pump, laser and/or recovery basket) by a single surgeon without having to interrupt the manipulation of the flexible endoscope.

Preferably, the endoscope holder has a translation unit on the side for translational movement of a tool inserted into the endoscope, the translation unit being positioned on the endoscope holder in such a way that it can be operated with the same hand when the endoscope is gripped by a surgeon. For example, the tool may be a retrieval basket for removing kidney stones.

Furthermore, the endoscope or the endoscope holder preferably has a lever for bending the endoscope tip, wherein the lever is connected to a position encoder via a transmission element, so that a state in which the endoscope tip is bent can be detected. This can prevent the endoscope from being pulled out of the patient's body when its tip is angled, for example, which would result in injury to the patient and/or damage to the endoscope.

The lever is preferably mounted on ball bearings so that improved feedback of the forces and torques occurring at the surgical site to the surgeon is possible with as little interference as possible.

Furthermore, the endoscope holder preferably has a capacitive sensor on its inside, which detects when a surgeon grips the endoscope holder, in particular switching between different operating modes thereby. For example, a switch can be made from StopIF (robot is switched on, but only holds its position) to cartImp_torIF (gravity-compensated movement of the robot arm and its payload is possible, wherein virtual fixtures can optionally be used to support the surgeon).

Furthermore, it is preferred that the translation unit limits the advance of a cylinder piston by means of a clamping force, wherein the clamping force is provided by two seals, which can be compressed in particular by means of a screw or a nut in such a way that the clamping force is increased as a result. In other words, the seal deter-mines how much force must be applied to perform a translation movement, i.e. how firmly the translation unit holds its position. This allows each surgeon to customize the operability of the translational advancement of the tool. In particular, the screw and/or nut can be operated by the surgeon without tools.

Furthermore, it is preferred that the pump cassette of the pump unit can be replaced without tools. The pump cassette can be a commercially available peristaltic pump. A flexible silicone tube can be positioned along the inside of the round pump housing. This is preferably pressed together using three rollers, which are rotatably mounted in relation to a roller carrier. The drive motor rotates the roller carrier (which in turn is rotatably mounted in relation to the pump housing) around its central axis and thus causes the rollers to roll on the silicone hose. The advantage of the cassette, which can be changed without tools, is the simplified compliance with sterility requirements: As the irrigation fluid enters the patient, it must be sterile. A fresh sterile cassette must therefore be fitted before each procedure.

It is also preferred that the surgical system has a fastening device for fastening the patient-side unit to the side rails of the operating table. This enables simple and secure attachment.

It is further preferred that the patient-side unit has a holding structure to which an auxiliary arm with a movable shaft and a clamp for holding a laser fiber or a safety wire is attached. This further improves the one-handed operability of the surgical system.

### BRIEF DESCRIPTION OF THE DRAWINGS

The terms Fig., Figs., Figure, and Figures are used interchangeably in the specification to refer to the corresponding figures in the drawings.

In the following, preferred embodiments of the invention are described by means of the figures, in which

The Figures show:

FIG. 1: Manual operation of a flexible ureteroscope

FIG. 2: Detailed view of the endoscope tip in the unbent and in the bent state

FIG. 3: Typical end effectors that are integrated into the working channel of flexible ureteroscopes

FIG. 4: Hardware architecture of a system for collaborative robotic endoscopy

FIG. 5: A modular software architecture of the system for collaborative robotic endoscopy

FIGS. 6 and 7: An embodiment of the robot-side unit

FIG. 8: Cross-section through the translation unit of the robot-side unit

FIG. 9: Position encoder for determining the lever position on the endoscope handle

FIG. 10: A flow chart for pump control using two buttons

FIG. 11: A pump unit for active rinsing

FIG. 12: An embodiment of the patient-side unit of the system according to the invention.

### DESCRIPTION OF THE INVENTION

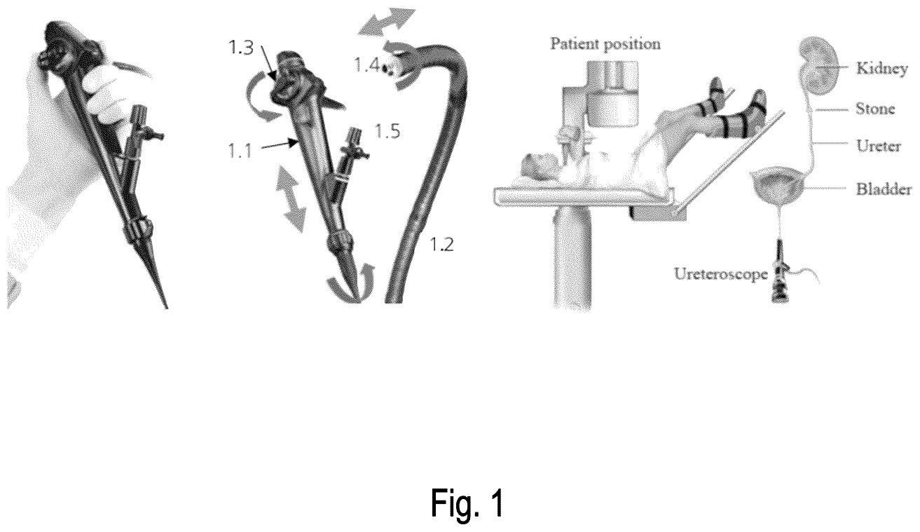

FIG. 1 shows the manual operation of a flexible ureteroscope (endoscope for urological procedures): The doctor holds the handle (1.1) in one hand (left illustration), the other hand guides the flexible shaft (1.2), usually near the access to the patient. The endoscope tip (1.4) can be bent in one plane by moving the actuating element (1.3). The plane in which the endoscope tip bends can be varied by rotating the entire endoscope around its longitudinal axis. The endoscope tip is advanced by translating the entire endoscope. Various tools such as an optical fiber for a laser or a retrieval basket for removing kidney stones can be inserted through the working channel (1.5).

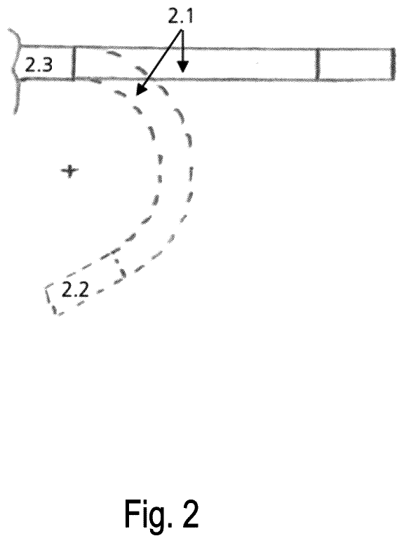

FIG. 2 shows a detailed view of the endoscope tip in the unbent state (solid lines) and in the bent state (dashed lines). The flexible area of the endoscope tip 2.1 is bent by actuating the adjusting wheel on the handle using cables/rods running in the endoscope shaft. The endoscope tip 2.2 and the rest of the shaft 2.3 remain rigid in the meantime.

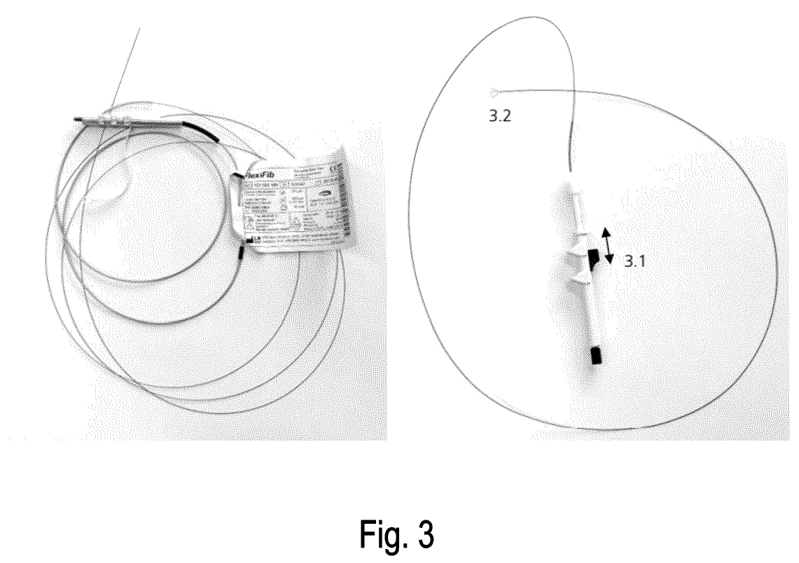

FIG. 3 shows typical end effectors that are integrated into the working channel of flexible ureteroscopes: Laser fiber for breaking up kidney stones (left), recovery basket for catching the stone debris (right). Translating the black handle element (3.1) in the direction of the arrow opens and closes the recovery basket (3.2) at the tip of the end effector.

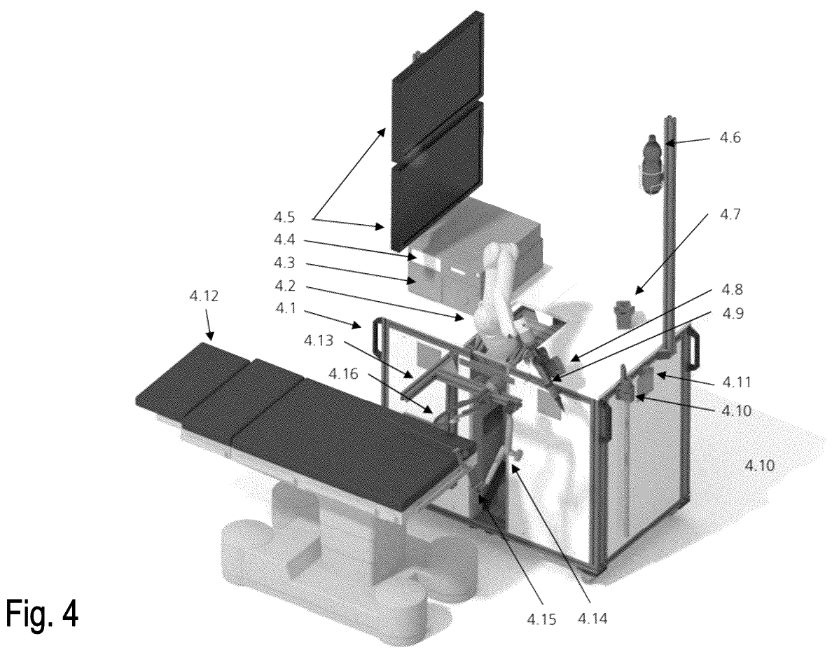

FIG. 4 shows a hardware architecture of the system for collaborative robotic endoscopy: The mobile carriage (4.1) contains the robotic arm (4.2) , the light source (4.3) and the video unit (4.4) of the flexible endoscope, monitors (4.5) for displaying the endoscope image and the graphic surface of the robotic system, PCs and the laser light source (inside the cart, not shown), liquid for irrigation (4.6), and a pump unit for active irrigation (4.7). Preferably, the robot base is height-adjustable and can be tilted in at least one axis. The robot-side unit (RSU; 4.8) with the flexible endoscope (4.9) is attached to the robot's tool interface. Optional brackets for the flexible endoscope (4.10) and the RSU (4.11) are attached to the mobile carriage. Intraoperatively, the mobile carriage is positioned near the operating table (4.1). The patient-side unit (PSU; 4.13) is located in the immediate vicinity of the patient. Attachment to the operating table is preferred, and attachment to the side rails of the operating table is particularly preferred. At least one retaining arm (4.14) for the airlock holder (4.15) is attached to the structure of the PSU. Preferably, at least one auxiliary arm (4.16) is also integrated into the PSU.

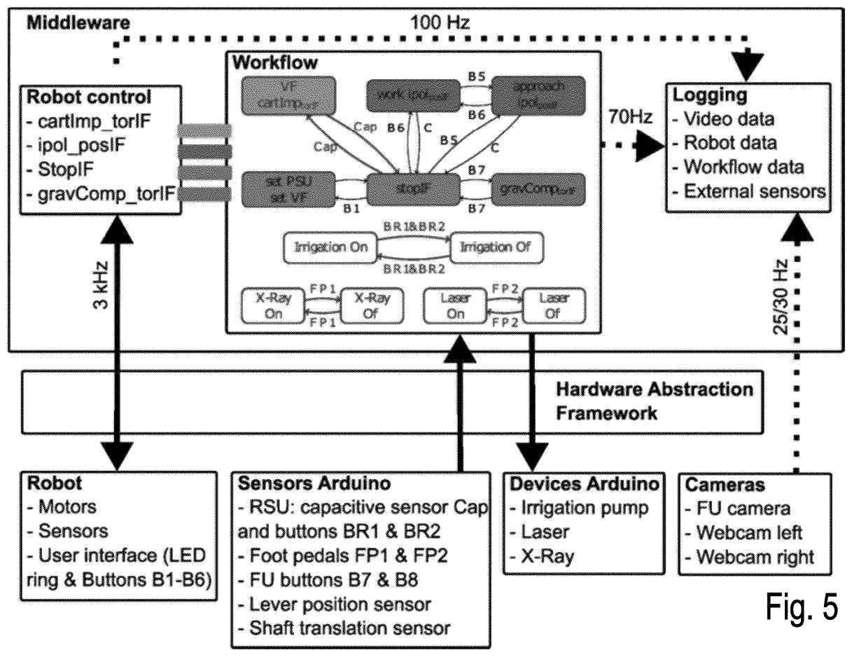

FIG. 5 shows a modular software architecture of the system for collaborative robotic endoscopy: The workflow can be triggered and parameterized externally and contains several state machines. A state machine activates and parameterizes the various control modes of the robot arm, for example a Cartesian impedance controller (car-tImp_torIF), a position controller (ipol_posIF), a stop controller (StopIF) and a force controller (gravComp_torIF). The robot controller communicates with the robot hardware (motors, sensors and user interface) via the Hardware Abstraction Framework at 3 KHz. Another state machine controls active flushing (irrigation). State machines 3 and 4 control the X-ray unit and the laser light source. The workflow can address external sensors or external active devices via microcontrollers (Arduino Micro in the example, whose firmware represents the hardware abstraction framework in this context). Data from various sources (robots, workflow, camera images) can be logged by a logging framework for documentation, fault diagnosis or research purposes. The middleware implements the communication between the high-level software components (robot control, workflow and logging).

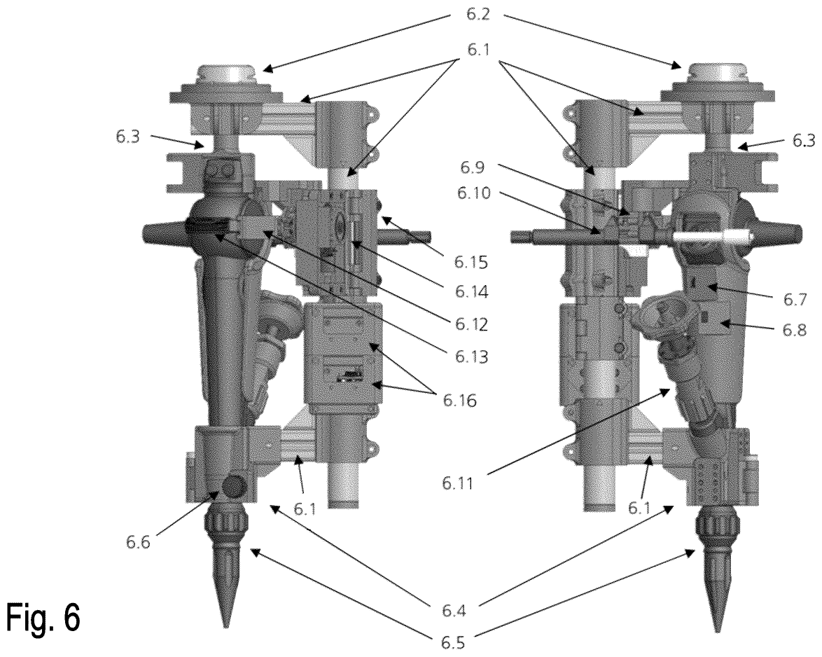

FIG. 6 shows a rendering of the robot-side unit (RSU): The RSU structure (6.1) connects the docking element for the robot tool interface (6.2) with the upper end of the endoscope handle holder (6.3). Preferably, the lower end of the endoscope handle holder (6.4) is also connected to the RSU structure in order to increase the stability of the endoscope head fixation. Preferably, the endoscope handle (6.5) can be fixed in the endoscope handle holder without tools, e.g. using a knurled screw (6.6). The endoscope handle holder has recesses for an upper button (6.7) and a lower button (6.8), as well as a clamp (6.9) for attaching the recovery basket handle (6.10). The translation unit (6.11) is attached to the working channel of the endoscope. A transmission element (6.12) transmits the movement of the lever for bending the endoscope tip (6.13) to a position encoder (6.14). The transmission element and encoder are rotatably connected to the RSU structure, which is round in this area, via a two-part terminal (6.15). One or more microcontrollers (6.16) process the signals from the buttons, the position encoder and the capacitive sensor attached to the inside of the endoscope handle holder before they are forwarded to the workflow.

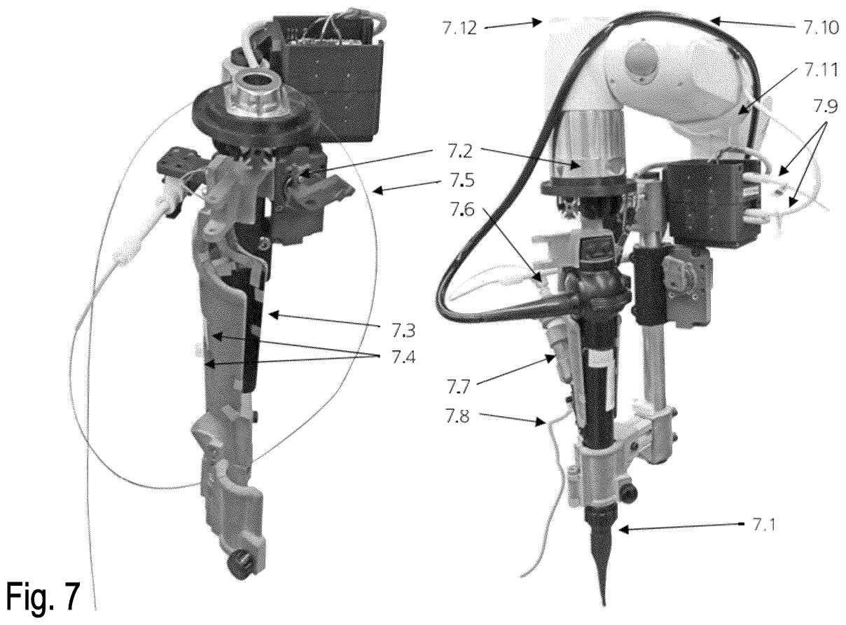

FIG. 7 shows the robot side unit (RSU) loose (left) and with flexible endoscope (7.1) attached to the tool interface of the robot arm (7.2) (right). The capacitive sensor on the inside of the endoscope handle holder (7.3) detects when the user grips the RSU, film buttons (7.4) on the outside of the endoscope handle holder allow external devices such as the pump unit for active irrigation to be controlled. The shaft of the recovery basket (7.5) or alternatively a laser fiber is connected to the movable piston of the translation unit (7.7) via a union nut (7.6). The tube for the irrigation fluid (7.8) is typically also connected to the entrance to the working channel of the endoscope, for example via a three-way valve as shown here. The cables of the microcontroller (7.9) and the flexible endoscope (7.10) are fixed to the robot structure using cable clamps, Velcro tape or similar (7.11). The LED ring on the robot's tool interface (7.12) informs the user about the system status.

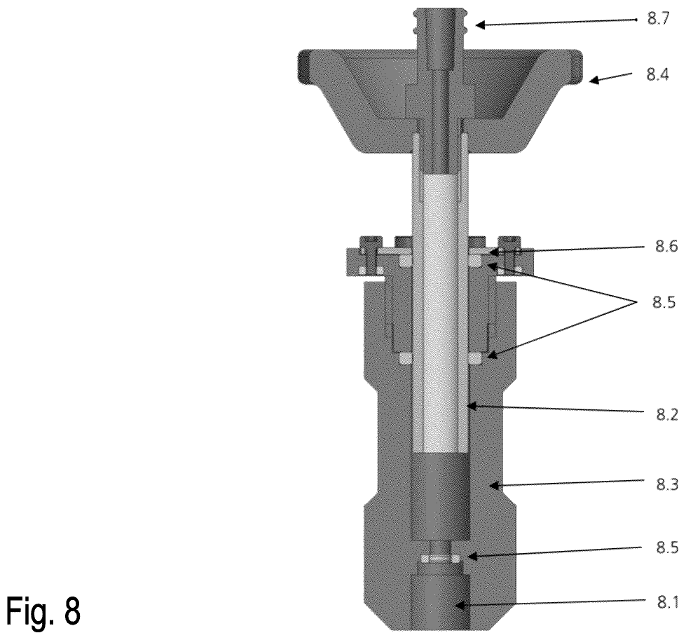

FIG. 8 shows a cross-section through the translation unit of the RSU: The translation unit is either screwed directly onto the working channel of the flexible endoscope or onto an intermediate piece with irrigation connection. The corresponding thread is located in the hole (8.1). The user can move the cylinder piston (8.2) relative to the structure of the translation unit (8.3), gripping it by the handle (8.4). The seals (8.5) prevent the flushing fluid from leaking. Preferably, the compression of the seals and thus the resistance when moving the piston can be varied via a screwed cover (8.6) or similar. A sealing element (e.g. O-ring) must be provided between the end effector and the structure to prevent leakage. A laser fiber or another end effector can be inserted and fixed to the Luer lock (8.7) using a union nut. Once the end effector is fixed, the user can move the end effector by moving the cylinder piston with one hand.

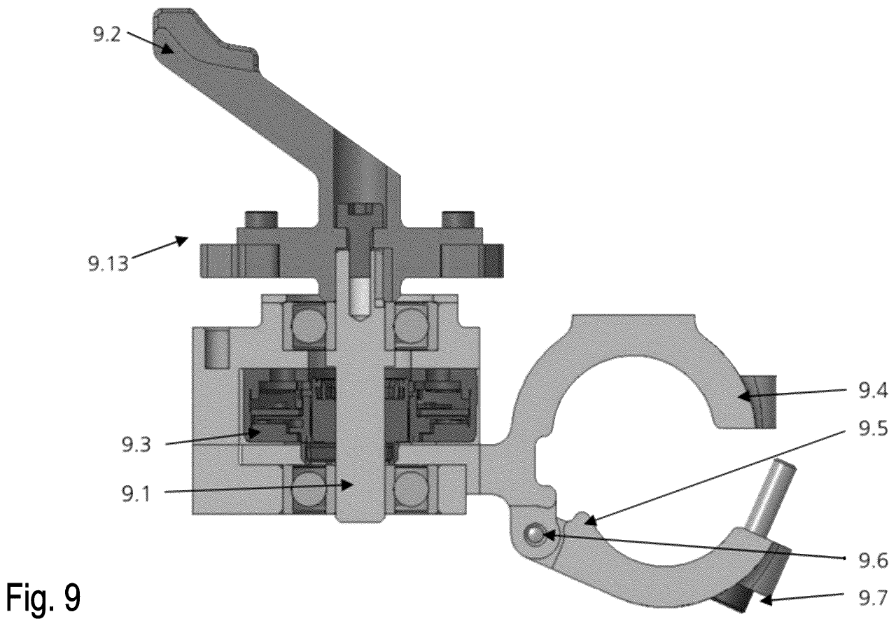

FIG. 9 shows a position encoder for determining the lever position on the endoscope handle A rotatably mounted shaft (9.1) and the transmission element (9.2) transmit the movement of the lever for bending the endoscope tip (not shown) to the position encoder (9.3). The two-part clamp (9.4) and (9.5) with the hinge pin (9.6) allows the encoder assembly to be rotated around the round RSU structure in this area. The encoder assembly is clamped by tightening at least one screw (9.7).

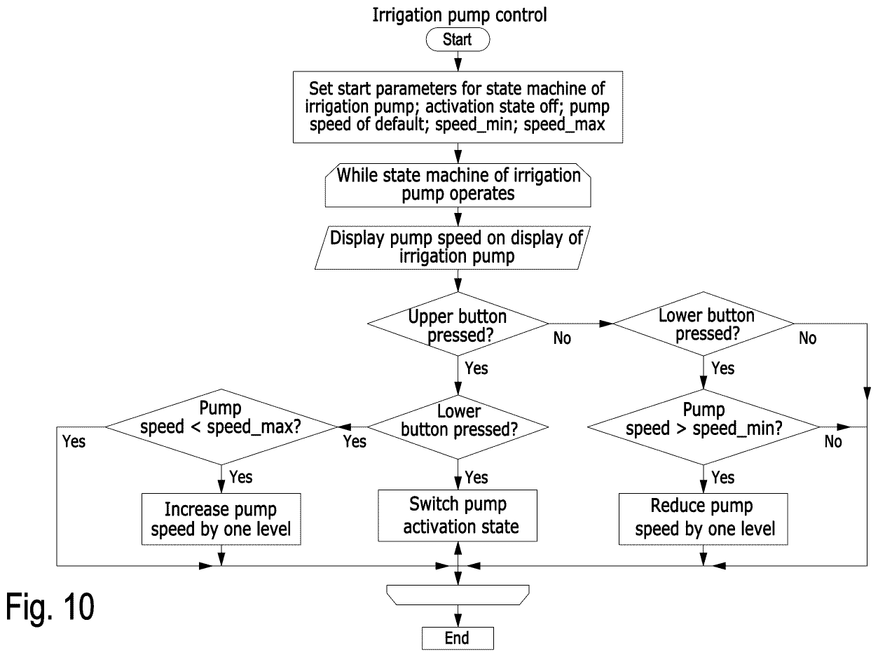

FIG. 10 shows a flow chart for pump control using two buttons After starting the program, the state machine is first parameterized with the stored default values for the switch-on state of the pump (off), the pump speed (default), the minimum pump speed (Speed_min) and the maximum pump speed (Speed_max). These default values are predefined and cannot be changed by the surgeon. A While loop then starts, which runs continuously as long as the state machine is active. Within the loop, the current pump speed is first shown on the display of the pump unit. The user inputs are then queried at the two buttons on the RSU. If only the upper button is pressed and the pump speed is lower than the maximum pump speed, the pump speed is increased by one level. If only the lower button is pressed and the pump speed is greater than the minimum pump speed, the pump speed is reduced by one level. If both buttons are pressed simultaneously, the switch-on status of the pump is changed (from off to on or vice versa). The loop then starts again. In order not to miss any user input during the program runtime, a flag can be set via an interrupt routine when a button is pressed, which is reset again after the user input has been processed.

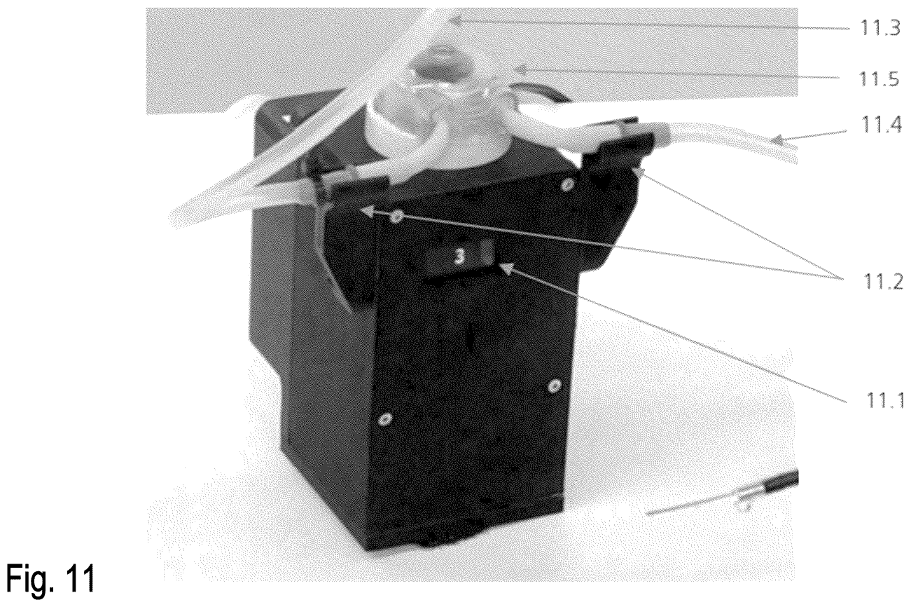

FIG. 11 shows a pump unit for active rinsing The pump unit has a screen for displaying the pump speed (11.1) and two clamps (11.2) for attaching the tubes to the fluid reservoir (11.3) and the flexible endoscope (11.4). The pump cassette (11.5) can be replaced without tools to ensure sterility.

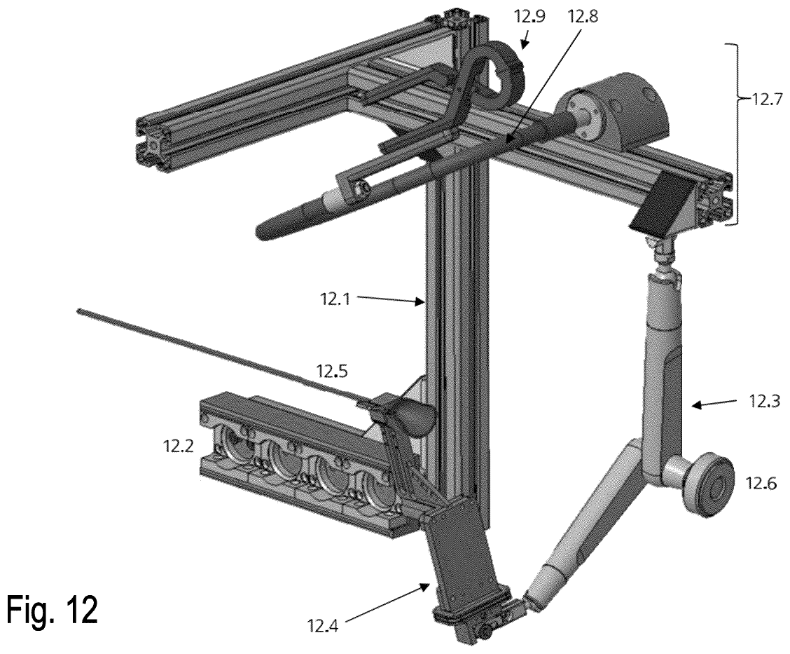

FIG. 12 shows a rendering of the patient-side unit (PSU) of the system for collaborative robotic endoscopy: The PSU structure (12.1) is attached to the side rails of the operating table using suitable fixings (e.g. clamps (12.2)). At least one holding arm (12.3) for the UAS holder (12.4) with the Ureteral Access Sheat (UAS) (12.5) is attached to the PSU structure. Preferably, this holding arm is movable in several degrees of freedom and can be easily locked (for example via the knurled screw 12.6). In addition, at least one auxiliary arm (12.7) consisting of a movable shaft (12.8) and a clamp (12.9) is attached to the PSU structure.

1. A surgical system for minimally invasive robotic surgery comprising:
a holding arm, having an endoscope holder for a handle unit of a conventional endoscope,
a patient-side unit that can be attached close to a patient and enables defined positioning of the patient access,
a liquid container, and
a pump unit for pumping an irrigation fluid into the patient's body.

2. The system of claim 1, further comprising an endoscope having at least one actuating button for actuating the pump unit and/or a laser.

3. The system of claim 1, wherein the endoscope holder includes a translation unit on a side for translational movement of a tool inserted into the endoscope, the translation unit being positioned on the endoscope holder in such a way that it can be operated with the same hand when the endoscope is gripped by a surgeon.

4. The system of claim 1, further comprising a lever for bending a tip of the endoscope, wherein the lever is connected via a transmission element to a position encoder, so that a state in which the endoscope tip is bent can be detected, wherein the lever is in particular mounted on ball bearings.

5. The system of claim 4, wherein the endoscope holder has a capacitive sensor on its inner side by means of which detection takes place when a surgeon grips the endoscope holder, whereby in particular switching between different operating modes takes place as a result.

6. The system of claim 3, wherein the translation unit can limit the advance of a cylinder piston by means of a clamping force, the clamping force being generated by two seals which can be compressed, in particular by means of a screw, in such a way that the clamping force is thereby increased.

7. The system claim 6, wherein the pump cassette of the pump unit can be replaced without tools.

8. The system of claim 1, further comprising a fastening device for fastening the patient-side unit to side rails of an operating table.

9. The system of claim 1, wherein the patient-side unit comprises a holding structure to which an auxiliary arm with a movable shaft and a clamp for holding a laser fiber is attached.
